(19) United States
(12) Patent Application Publication
(10) Pub. No.: US 2010/0256489 A1
(43) Pub. Date: Oct. 7, 2010

(54) Handheld Transducer Scanning Speed Guides and Position Detectors

(75) Inventors: Laust G. Pedersen, Santa Barbara, CA (US); Constantine C. Davlantes, Carpinteria, CA (US)

Correspondence Address: BLAKELY SOKOLOFF TAYLOR & ZAFMAN LLP, 1279 OAKMEAD PARKWAY, SUNNYVALE, CA, 94085-4040, US

(73) Assignee: NivaSonix, LLC, Carpinteria, CA (US)

(21) Appl. No.: 12/680,538

(22) PCT Filed: Sep. 29, 2008
(86) PCT No.: PCT/US08/78188

§ 371 Date: Mar. 26, 2010

Related U.S. Application Data
(60) Provisional application No. 60/995,895, filed on Sep. 28, 2007.

(51) Int. Cl. A61N 7/00; A61B 8/00

(52) U.S. Cl. 600/439

(57) ABSTRACT

Methods for controlling and monitoring speed and position of a handheld medical transducer. Three methods are presented of various means, two of which include the user in the feedback loop and the third is fully automatic. In the third, an optical position sensor similar to an optical computer mouse provides enough information that the system can respond to and correct for a freehand scanning motion by the user.

### CROSS-REFERENCE TO RELATED APPLICATION

This application claims the benefit of U.S. Provisional Patent Application No. 60/995,895 filed Sep. 28, 2007.

### BACKGROUND OF THE INVENTION

1. Field of the Invention

The present invention relates to the field of non-invasive external ultrasound lipoplasty, skin tightening, and various non-invasive aesthetic, dermatologic, and therapeutic applications.

2. Prior Art

During a non-invasive external ultrasound lipoplasty, skin tightening, aesthetic, dermatologic, or other therapeutic procedure with a handheld transducer, it becomes particularly important to apply and distribute the ultrasound energy dose according to the amount and location of the fat to be emulsified, the degree and location of skin tightening needed, or the extent and type of aesthetic, dermatologic, or other therapeutic desired effect.

For this purpose the transducer's movement or instantaneous scanning speed needs to be known or better yet its position from which the scanning speed can be easily derived.

U.S. Pat. No. 7,347,855 teaches a passive system of computerized tracking of a multiplicity of target volumes with compensation for body movements.

U.S. Pat. No. 6,645,162 teaches an active tracking system depicted in their FIGS. 12 and 13 guiding a transducer in a linear motion.

“Selective Creation of Thermal Injury Zones in the Superficial Musculoaponeurotic System Using Intense Ultrasound Therapy”, (Matthew W. White et al., ARCH FACIAL PLAST SURG, Vol. 9, January/February 2007) shows an ultrasound probe by Ulthera with an internal transducer performing a controlled linear motion, thereby acting as an active tracking system.

U.S. Pat. No. 7,150,716 is specific to diagnostic ultrasound, and teaches two methods (and systems) of detecting transducer scanning speed, namely one through the use of an sensor similar to an optical computer mouse and another through real time de-correlation of ultrasound images.

In the case of a handheld transducer without hardware to do the spatial and time feedback, the control has to come through the operator. It should be realized that the optimum speed of the transducer across the skin is strongly related to the optimum local exposure time. Too short an exposure time (fast motion) will not give adequate cavitation or heat (when needed) and could therefore reduce the efficacy of the procedure to near zero. Too slow a motion could create too much cavitation or heat with the potential for indiscriminant tissue destruction. In order to separate cavitation and heating further, there may be cases where multiple passes over the same area are necessary.

### BRIEF DESCRIPTION OF THE DRAWINGS

FIG. 1a is a functional block diagram of one embodiment of the present invention.

FIG. 1b is a functional block diagram of an alternate form of the embodiment of FIG. 1a.

FIG. 2a is a functional block diagram of another embodiment of the present invention.

FIG. 2b is a functional block diagram of alternate form of the second embodiment.

FIG. 3 is a functional block diagram of still another embodiment of the present invention.

FIG. 4 is a view of a graphical user interface (e.g., touch screen display menu) with a scanning speed indicator in accordance with the embodiment of FIG. 1a.

FIG. 5 is a transducer with an integral scanning speed indicator, which in this example consists of five LEDs, in accordance with the embodiment of FIG. 1b.

FIG. 6 is a flexible scanning speed guidance strip with built in LEDs in accordance with the embodiments of FIGS. 2a and 2b.

FIG. 7 is a cross sectional view of an exemplary optical sensor in accordance with still another embodiment of the present invention.

### DETAILED DESCRIPTION OF THE PREFERRED EMBODIMENTS

The optimum transducer “scanning” speed for delivering a predetermined dose of ultrasound to a desired treatment area determined by a scanning plan is a function of both the cavitation related Mechanical Index (MI) and tissue temperature Thermal Index (TI) settings. While cavitation is a threshold mechanism there is both an amplitude factor beyond the threshold level and an exposure time factor involved in emulsifying a certain fraction of the treated fat, whereby low settings require a slow scanning speed and high settings require faster scanning speeds. The relationships can be estimated from the numerical values of MI and TI and further refined empirically using data from animal and clinical studies. Furthermore, since the transducer is not 100% energy efficient, its face (skin contact area) will create heat and if not properly controlled may present a hazard for potential skin burns. Moving the transducer across the skin surface will also significantly reduce localized peak skin temperature. In the case where there is a sensor monitoring the transducer face temperature, control of the skin heating can be included in the speed indicator. If there is no transducer face temperature sensor the suggested transducer movement speed component due to tissue heating can be based on empirical data from animal and clinical studies.

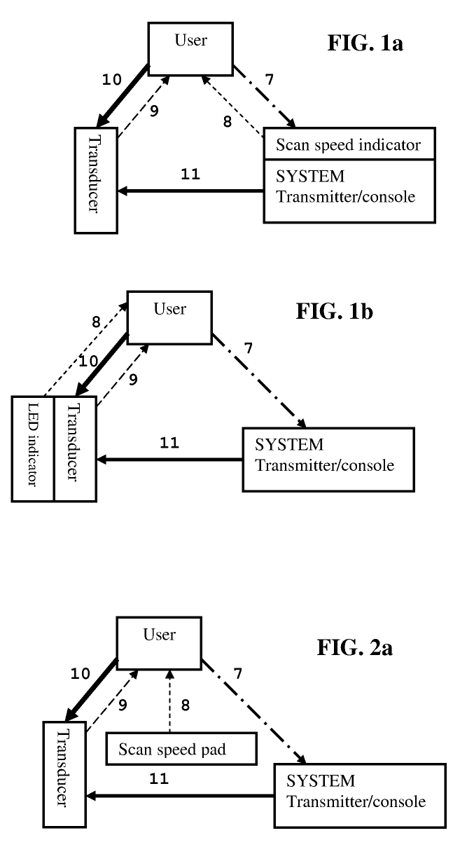

There are at least three approaches for addressing control of the ultrasound dose delivery, as summarized in FIGS. 1a and 1b, FIGS. 2a and 2b, and FIG. 3.

One method requires the user to be part of the feedback loop, whereby the system, the transducer, or a separate device acts as a visual guide for the user to apply the desired scanning speed. The first embodiment is an example of this approach.

Another method consists of a subsystem that detects the transducer scanning velocity and in real time transfers this information to the system, which in turn adjusts parameters such as MI and TI (i.e., ultrasound dose) to achieve the desired effect based on the actual speed of transducer movement. This relieves the user from precisely matching the desired scanning speed, but still requires the user to keep track of the transducer position and the ultrasound beam focal depth. The second embodiment is an example of this.

A third method monitors the transducer position, which is transferred to the system in real time. With this information and the presence of a clock, the transducer velocity may also be easily calculated. Now the system can automatically adjust the needed parameters such as MI, TI and focal length to accomplish the planned treatment, giving the user the freedom to move the transducer almost “at will”. The third embodiment is an example of this.

It should be noted that there is much more value in using a 3D coordinate system, where the (contoured) skin defines two of the dimensions and the depth below the skin surface is the third, rather than a Cartesian coordinate system fixed to the operating room or even fixed to localized patient movements.

The connecting lines in the functional block diagrams in FIGS. 1a to 3 have the following meaning. The occasional user control of the system/console is shown as 7. Item 8 indicates that the user reads the scanning speed indicator. Item 9 indicates that the user views the actual scanning speed of the transducer. Item 10 indicates that the user actually holds and moves the transducer. Item 11 shows the transmit signals from the system to the transducer. Item 12 shows the low level power supply and control signals from the system to the Scanning Speed Pad 5 or optical sensor. Item 13 indicates the path of sensor signals from the optical sensor to the built in Decoder, which translates the information into position and speed.

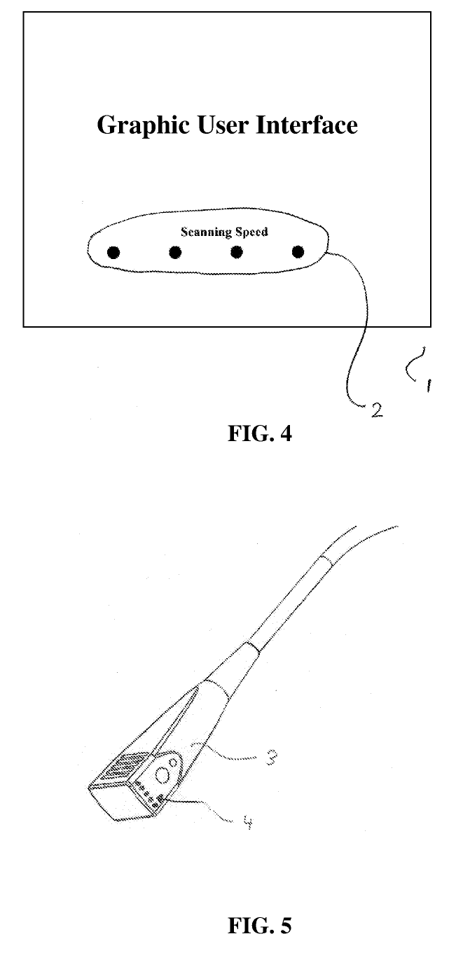

One embodiment (FIGS. 1a and 4) is to have a visual transducer scanning speed indicator on the transmitter (main unit, console or system) that moves with the same speed that is optimal for the transducer motion on the skin. The speed indicator can take the form of a moving cursor 2 on a screen 1 of the transmitter. The moving cursor can take many forms, the one shown in FIGS. 1a, 1b, 2a, 2b and 4 through 6 consists of four moving dots (light sources such as LEDs). These may be sequentially switched on and off at a controlled rate, or the first switched on, the second switched on, etc. with all being switched off and the cycle repeated after the last light source has been switched on, either of which is to be considered sequential switching on, or scanning. The user can then practice matching that speed while holding the transducer near the screen. When sufficiently proficient he/she can match the speed when scanning on the skin. As verification, the user can mark the skin for a certain distance and calculate the time needed to traverse that distance based on the numerical value of the desired velocity.

Another version (FIGS. 1b and 5) of the first embodiment is to have a visual transducer scanning speed indicator on the transducer itself 3, for example in the form of an array of visual indicators (light sources) such as Light Emitting Diodes (LEDs) 4, which light up in a sequence corresponding to the desired speed of the transducer. It will then be up to the user to provide the “feedback loop” by moving the transducer at the indicated speed. Here the user can perform the same verification as described above.

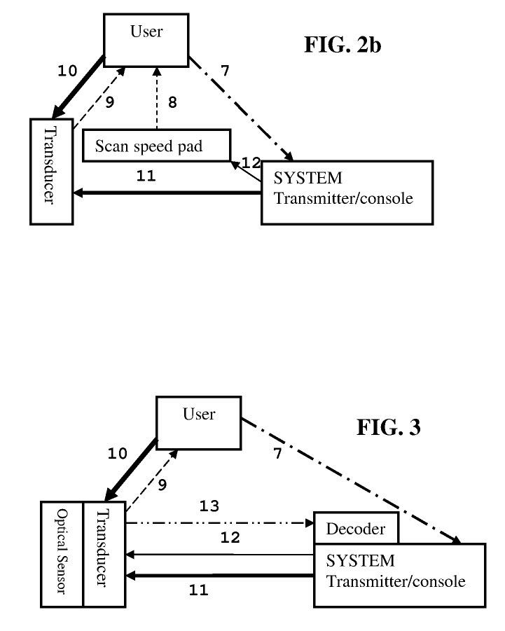

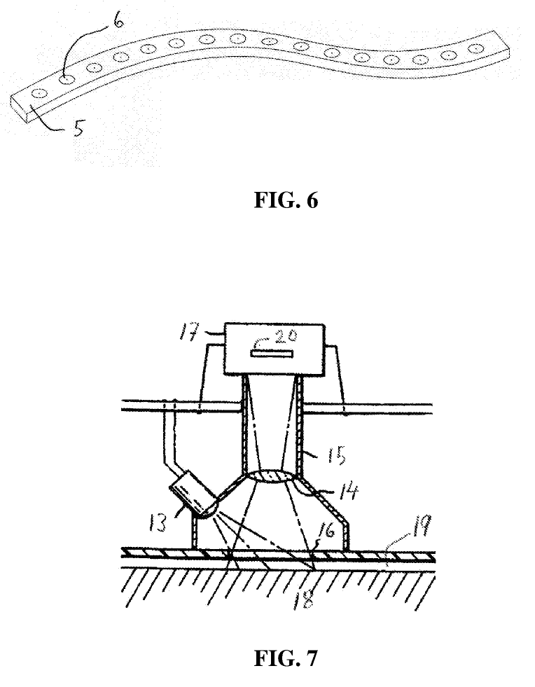

A second embodiment is a separate flexible scanning speed guidance pad 5 (FIGS. 2a, 2b and 6), which can be placed on the patient adjacent to the intended transducer path. The flexible material can be silicone rubber or other material with LEDs (or other visual indicators) 6 molded in. The LEDs are sequentially switched on and off so that they provide visual scanning speed guidance in proximity to the transducer. The speed guidance pad can be manufactured in different lengths and/or from different materials to fit the desired treatment area, and can also be either disposable (single patient use), semi-disposable, or reusable. The LEDs can be powered either by batteries, as in the embodiment of FIG. 2a, or by the transmitter, embodiment of FIG. 2b, in which case a power cord is detachably connected to the guidance pad. By being connected to the transmitter, the system may display the scanning speed, scanning location or position, and range of scan distance if the pad is physically longer than the desired scan needed to match the system settings, while the battery operated solution either would require wireless transmission of the information, or require the user to set the scanning speed according to the transmitter's displayed parameters.

A third embodiment is an optical 2D location sensor technology similar or identical to those used in an optical computer mouse, as in FIGS. 3 and 7. The sensor primarily consists of a light source 13, a translucent membrane or cavity 16, a lens 14 to collimate the reflected light from the skin 18, which also goes through the acoustic coupling gel 19 and continues through an optical guide to an optical sensor array 20 embedded in an integrated circuit 17. As indicated in FIG. 3 the optical sensor is attached to or built into a transducer, generally like that of FIG. 5. The sensor information is passed through the transducer cable and processed in the system to find the position and velocity of the transducer. Any speckle, phase shift, frequency shift or other characteristics may be used to detect motion and velocity.

Alternatively the sensor information may be wirelessly communicated to the system.

As with a computer mouse, the optical 2D location sensor can lose track of the transducer position if lifted from the surface (skin). This can be overcome with a simple calibration process, whereby the user moves the transducer to a marked calibration spot on the skin, push a calibration button on the transducer or on the system, and moves the transducer on the skin to the desired location.

In the case of a “brush-beam” (non circular symmetric beam) it becomes important to scan approximately perpendicular to the width (long axis) direction of the brush-beam.

This third embodiment is very adaptable to a scanning plan in which the user graphically composes a 3D volume using software within the system or off line, showing the relative location and amount of treatment wanted, both with respect to cavitation (fat emulsification), heating (skin tightening), or other aesthetic/dermatologic/therapeutic treatments. Off line use of the scanning plan software allows data transfer to the system. During the procedure, the system can keep track of the transducer's location and in real time can adjust critical parameters such as MI, TI and focal depth (if equipped with electronic focusing), so the desired treatment “dose” eventually will be delivered. The real time difference between the desired and actual delivered “dose” can also be displayed on the system graphically in a 2D format, so the user can concentrate the transducer motion in the area where more treatment is needed. This allows the user to move the transducer freely within certain boundaries with respect to both position and speed.

For the best outcome with respect to the treatment plan, the transducer needs to be oriented perpendicular to the skin and in the case of a brush-beam transducer, the scanning velocity vector needs to be perpendicular to the brush width direction. However, an angular error relative to the exact perpendicularity is a cosine function, meaning that it is a weak dependency, so that in reality, perpendicularity need not be monitored, but can be continuously estimated by the user.

The suggested speed shown by the various embodiments of the speed indicator can be based on MI, TI and instantaneous transducer face temperatures and/or acquired data from animal and clinical studies. While the above methods are intended to be used in conjunction with a non-invasive ultrasound lipoplasty transducer, the inventions, the scanning light source of the first two embodiments can be used on handheld transducers for other modalities, including aesthetic, dermatologic, or other therapeutic applications. In the claims to follow, a reference to a handheld external ultrasound treatment transducer is a reference to a handheld external ultrasound transducer useable for lipoplasty, skin tightening, aesthetic, dermatologic/, and other therapeutic purposes.

Thus, while certain preferred embodiments of the present invention have been disclosed and described herein for purposes of illustration and not for purposes of limitation, it will be understood by those skilled in the art that various changes in form and detail may be made therein without departing from the spirit and scope of the invention.

What is claimed is:

1. Apparatus for guiding the scanning speed of a user moving a handheld external ultrasound treatment transducer across a body comprising:
a plurality of light sources spaced apart in a row;
a light source driver for sequentially illuminating the light sources;
the light sources being disposed to be viewable by the user of the external ultrasound treatment transducer for visual reference of the speed at which the light appears to progress along the row.

2. The apparatus of claim 1 further comprised of user controlled input apparatus for varying the speed at which the light appears to progress along the row.

3. The apparatus of claim 1 wherein the plurality of light sources are disposed on an external ultrasound treatment system console coupled to a handheld external ultrasound treatment transducer.

4. The apparatus of claim 1 wherein the row of light sources are disposed on a handheld external ultrasound lipoplasty transducer and aligned generally in the direction of intended scanning motion of the handheld external ultrasound treatment transducer.

5. The apparatus of claim 1 wherein the row of light sources are disposed on a flexible pad for placing on a patient's body adjacent to an area of the patient's body intended to be scanned or treated by the ultrasound treatment transducer.

6. Apparatus for guiding the scanning speed a user moves a handheld device across a body comprising:
a plurality of light sources spaced apart in a row;
a light source driver for sequentially illuminating the light sources;
the light sources being disposed to be viewable by the user for visual reference of the speed at which the light appears to progress along the row.

7. The apparatus of claim 6 further comprised of user controlled input apparatus for varying the speed at which the light appears to progress along the row.

8. The apparatus of claim 6 wherein the row of light sources are disposed on a handheld device and aligned generally in the direction of intended scanning motion of the handheld device.

9. The apparatus of claim 6 wherein the row of light sources are disposed on a flexible pad for placing on a patient body adjacent to an area of the patient's body intended to be scanned or treated by the handheld device.

10. Apparatus comprising:
an optical sensor for sensing relative position and velocity while a user moves a handheld external ultrasound treatment transducer across a patient's body having;
a handheld external ultrasound lipoplasty transducer for scanning across a patient's body;
a light source in the handheld external ultrasound lipoplasty transducer disposed to illuminate skin of the patient's body;
a lens disposed to collimate light reflected from the skin;
an optical sensor array disposed to receive the light collimated by the lens;
an output of the optical sensor array being coupled through a cable associated with the handheld external ultrasound lipoplasty transducer or coupled wirelessly to a system console; and,
the system console being configured to process the output of the optical sensor array.

11. A method for guiding the scanning speed a user moves a handheld external ultrasound lipoplasty transducer across a body comprising:
providing a plurality of light sources spaced apart and disposed in a row viewable by the user of the external ultrasound lipoplasty transducer;
sequentially switching the light sources on at a rate selected to appear to progress at a desired scanning speed.

12. The method of claim 11 further comprising varying the speed at which the light appears to progress along the row.

13. The method of claim 11 further comprising:
disposing the plurality of light sources on an external ultrasound treatment system console, whereby a user may practice the desired scanning speed.

14. The method of claim 11 further comprising:
disposing the plurality of light sources on the handheld external ultrasound treatment transducer.

15. The method of claim 11 further comprising:
disposing the row of light sources on a handheld external ultrasound treatment transducer and aligned in the direction of intended scanning motion of the handheld external ultrasound lipoplasty transducer;
whereby the user may use the sequential switching of the light sources as a scanning speed guide.

16. The method of claim 11 further comprising:
disposing the row of light sources on a flexible pad for placing; and,
laying the flexible pad on a patient's body adjacent to an intended scan area of the patient's body;
whereby the user may use the sequential switching of the light sources as a scanning speed guide.

17. A method for guiding the scanning speed a user moves a handheld device across a body comprising:
providing a plurality of light sources spaced apart and disposed in a row viewable by the user of the external ultrasound treatment transducer;
sequentially switching the light sources on at a rate selected to appear to progress at a desired scanning speed.

18. The method of claim 17 further comprising varying the speed at which the light appears to progress along the row.

19. The method of claim 17 further comprising:
disposing the plurality of light sources on the handheld device.

20. The method of claim 17 further comprising:
disposing the row of light sources on a handheld device and aligned in the direction of intended scanning motion of the handheld device;
whereby the user may use the sequential switching of the light sources as a scanning speed guide.

21. The method of claim 17 further comprising:
disposing the row of light sources on a flexible pad for placing; and,
laying the flexible pad on a patient's body adjacent to an intended scan area of the patient's body;
whereby the user may use the sequential switching of the light sources as a scanning speed guide.

22. A method for detecting relative position and scanning speed of a handheld external ultrasound treatment transducer with a system providing real-time parameter adjustment to adhere to a treatment plan during free-hand scanning across a body comprising:
an optical sensor for sensing motion and velocity when a user moves a handheld external ultrasound treatment transducer across a patient's body attached to;
a handheld external ultrasound treatment transducer for scanning across a patient's body;
an output of the optical sensor array being coupled through a cable associated with the handheld external ultrasound treatment transducer or coupled wirelessly to a system console;
the system console being configured to process the output of the optical sensor array; and,
the system console also being configured to adjust critical acoustic parameters in real time based on the detected transducer position and scanning speed in order to adhere to a procedure plan.
